(19) United States
(12) Patent Application Publication
(10) Pub. No.: US 2007/0032810 A1
(43) Pub. Date: Feb. 8, 2007

(54) Medical cutting and/or holding instrument

(76) Inventor: Martin Storz, Tuttlingen (DE)

Correspondence Address: ST. ONGE STEWARD JOHNSTON & REENS, LLC, 986 BEDFORD STREET, STAMFORD, CT, 06905-5619, US

(21) Appl. No.: 11/509,153

(22) Filed: Aug. 24, 2006

Related U.S. Application Data
(63) Continuation of application No. PCT/EP05/01804, filed on Feb. 22, 2005.

(30) Foreign Application Priority Data
Feb. 25, 2004 (DE) 10 2004 009 199.4

(51) Int. Cl. A61B 17/32

(52) U.S. Cl. 606/174

(57) ABSTRACT

The invention relates to a medical cutting and/or holding instrument having a shaft, a handle consisting of two gripping members on the proximal end of the shaft, and a push-pull rod for activating a tool that is positioned on the distal end of the shaft and consists of two jaw members, such that the push-pull rod can be coupled with at least one rotatable jaw member of the tool on the distal end and with one movable gripping member of the handle on the proximal end. To produce a medical cutting and/or holding instrument that is of simple construction and allows simple and versatile handling, it is proposed with the invention that the push-pull rod is configured as a component that is independent of the shaft and mounted outside the shaft, entirely on the rotatable jaw member of the tool and on the movable gripping member of the handle.

### CROSS-REFERENCE TO RELATED APPLICATIONS

The present application is a continuation of pending International patent application PCT/EP2005/001804 filed on Feb. 22, 2005 which designates the United States and claims priority from German patent application 10 2004 009 199.4 filed on Feb. 25, 2004, the content of which is incorporated herein by reference.

### FIELD OF THE INVENTION

The invention relates to a medical cutting and/or holding instrument having a shaft, a handle consisting of two gripping members on the proximal end of he shaft, and a push-pull rod for activating a tool that is positioned on the distal end of the shaft and consists of two jaw members, such that the push-pull rod can be coupled with at least one rotatable jaw member of the tool on the distal end and with one movable gripping member of the handle on the proximal end for opening and closing the tool.

### BACKGROUND OF THE INVENTION

Medical cutting and/or holding instruments are widely employed both in open surgery and in endoscopic surgery. Because the nature of their use demands a very slender construction for endoscopic instruments, which as a rule are inserted through hollow trocar sleeves into the surgical area, in endoscopic surgery so-called tubular shaft instruments have established and proved themselves for the configuration of cutting and/or holding instruments. These tubular shaft instruments are distinguished in that the instrument shaft is configured as a hollow tube and the push-pull rod provided for activating the distal-end jaw member is positioned so that it can be slid along the interior of the hollow shaft tube. Because of this miniaturizing of the push-pull rod, required by the construction, and the dual-sided jointed mechanisms, tubular shaft instruments of this kind are not suited for use in open surgery, in which it is frequently necessary to transmit considerable force by means of the push-pull rod.

In addition, the hollow instrumental shafts of these instruments can only be cleaned with difficulty and require a complex structure for dismantling the instruments.

A generic medical instrument for open surgery is known, for instance, from DE 195 13 572 C2. In this familiar surgical instrument the push-pull rod is mounted in slit channels on the instrumental shaft. This construction allows the use of massive forceps profiles in configuring the shaft as well as the push-pull rod, so that great force can also be transmitted by the handle to the distal-end tool.

The disadvantage of this known construction, however, is that the push-pull rod and the shaft are coupled to one another by the slit channels and thus the two components must be precisely adapted to one another. First, this requires a precise technical manufacturing adaptation of the components that are to be coupled to one another and, secondly, it clearly limits flexibility for using the instrument for other purposes.

It is consequently the aim of the invention to design a medical cutting and/or holding instrument of the aforementioned type in such a way that, along with simple construction, it makes possible a simple and versatile operation.

### SUMMARY OF THE INVENTION

This aim is fulfilled by means of the invention in that the push-pull rod is configured as a component that is independent of the shaft and that is mounted outside the shaft, exclusively on the rotatable jaw member of the tool and on the movable gripping member of the handle.

Because of the complete separation of the components—the shaft and push-pull rod—according to the invention, it becomes possible for the first time to configure both components independently of one another precisely as required by the particular intended use. In addition to allowing easy and thorough cleaning of the two separate components, this design has the advantage that the shaft and/or the push-pull rod are individually replaceable for the purposes of adapting the instrument.

According to a practical embodiment of the invention, it is possible to expand the usefulness of a medical instrument designed according to the invention for a range of surgical purposes because the push-pull rod and the shaft are of distinct configuration in terms of shape and/or length.

To allow an ergonomically favorable operation even in narrow and deep surgical locations, it is further proposed with the invention that the push-pull rod and/or the shaft has at least one bend or curvature with respect to its longitudinal axis.

According to a practical embodiment of the invention it is proposed that a breech is made in the shaft or in the push-pull rod for passage of the other respective component.

For example, according to another embodiment of the invention, at least one channel is configured running in the axial direction in or on the shaft and/or the push-pull rod to receive lighting wires and/or to form irrigation and suction channels for liquid and gas media.

Work on deeply situated surgical access spots, according to the invention, can be further facilitated by an illumination element, for instance with internal power supply, that can be secured on the shaft or on the push-pull rod.

According to a preferred embodiment of the invention it is proposed that the push-pull rod on the proximal end can be dissolubly secured on the movable gripping member by a coupling mechanism. This dissoluble coupling onto the movable gripping member clearly facilitates the cleaning and simplifies the assembly and disassembly of the instrument. This coupling mechanism preferably takes the form of a screw-in connection, so that it is possible, for instance, to screw the push-pull rod directly into the movable gripping member to form this screw-in connection, or else to connect the two components to one another by means of a screw-in threaded bolt.

It is further proposed with a first embodiment of the invention that the movable gripping member of the handle takes the form of a gripping member that can rotate around a pivot axis, so that the pivot axis, around which the gripping member can rotate, can be mounted either on the shaft or on the rigid gripping member of the handle.

According to an alternative second embodiment of the invention it is proposed that the movable gripping member of the handle is configured as a gripping member that can be slid axially in the direction of the push-pull rod.

The number of total components to be installed can be reduced, according to the invention, if the non-movable gripping member is configured as a one-piece unit with the shaft.

Finally, it is proposed with the invention that a support independent of the movable gripping member is configured on the handle for the user's index finger. This index finger support, independent of the movable gripping member, in addition to supporting the hand on the rigid gripping member of the handle, allows a gentle, stable activation of the medical instrument.

Further characteristics and advantages of the invention can be seen with the support of the accompanying illustrations, in which six embodiments of a medical cutting and/or holding instrument according to the invention are depicted in schematic, exemplary form.

### BRIEF DESCRIPTION OF THE DRAWINGS

FIG. 1a shows a partly cut-out side view of a first embodiment of a medical cutting and/or holding instrument according to the invention.

FIG. 1b shows an alternative embodiment of detail 1b from FIG. 1a.

FIG. 2 shows a partly cut-out side view of a second embodiment of a medical cutting and/or holding instrument according to the invention.

FIG. 3 shows a partly cut-out side view of a third embodiment of a medical cutting and/or holding instrument according to the invention.

FIG. 4 shows a partly cut-out side view of a fourth embodiment of a medical cutting and/or holding instrument according to the invention.

FIG. 5 shows a side view of a fifth embodiment of a medical cutting and/or holding instrument according to the invention.

FIG. 6 shows a partly cut-out side view of a sixth embodiment of a medical cutting and/or holding instrument according to the invention.

### DETAILED DESCRIPTION OF THE INVENTION

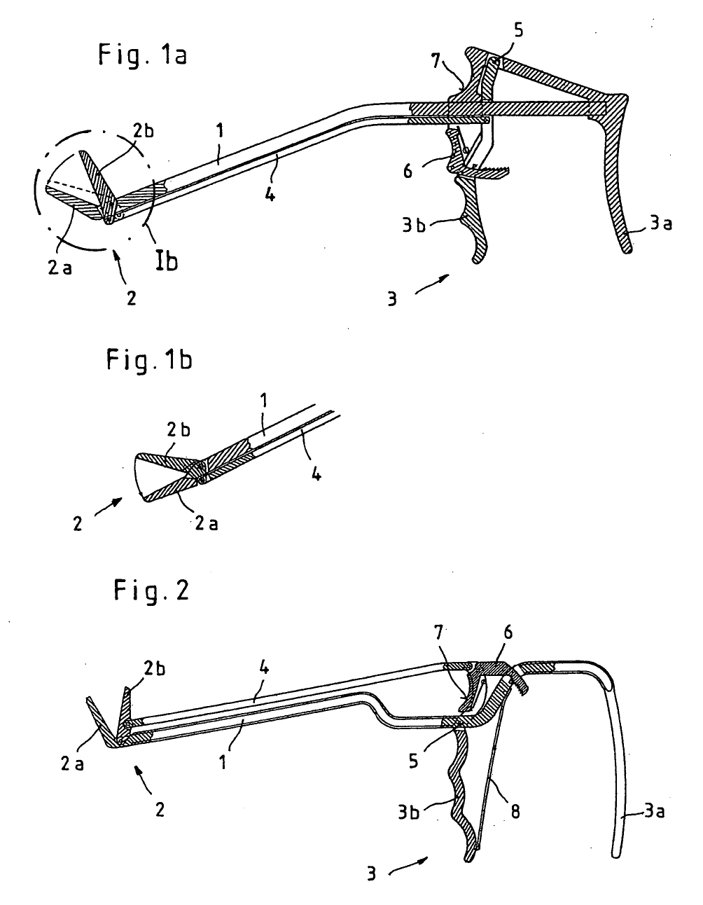

The medical cutting and/or holding instruments depicted in FIGS. 1a and 2 to 6 consist essentially of a stalk-shaped shaft 1, a tool 2 that is positioned on the distal end of the shaft 1 and consists of two jaw members 2a and 2b, a handle 3 positioned on the proximal end of the shaft and equipped with two gripping members 3a and 3b, and a push-pull rod 4 that can be slid axially for activating the tool 2 by means of the handle 3.

As can further be seen from the illustrations, the tool 2 consists of a rigid jaw member 2a firmly connected with the shaft 1 and a jaw member 2b that can rotate by means of the push-pull rod 4. The handle 3 likewise has a rigid gripping member 3a that is firmly connected with the shaft 1 as well as a gripping member 3b that can move with respect to the rigid gripping member 3b. The rotatable jaw member 2b and the movable gripping member 3b are coupled to one another by means of the push-pull rod 4, so that the push-pull rod 4 and the rotatable gripping member 3b can be dissolubly connected to one another by a coupling mechanism in such a way that the push-pull rod 4 can be rotated upward away from the shaft 1, for instance for purposes of cleaning.

While the movable gripping member 3b can be rotated around its pivot axis 5 with respect to the rigid gripping member 3a in the medical instruments depicted in FIGS. 1a to 4, FIGS. 5 and 6 show an embodiment in which the rotatable gripping member is configured so that it can slide axially in the shaft direction.

By configuring the push-pull rod 4 as a self-sufficient component, independent of the configuration of the shaft 1, and positioned outside the shaft 1, exclusively on the rotatable jaw member 2b of the tool 2 and on the movable gripping member 3b of the handle 3, both components 1 and 4 can be configured so that they are individual and adapted to the particular use. The components 1 and 4 can also take the form of massive shafts, as required for use in open surgery, by which even strong forces can be transmitted from the handle 3 to the tool 2.

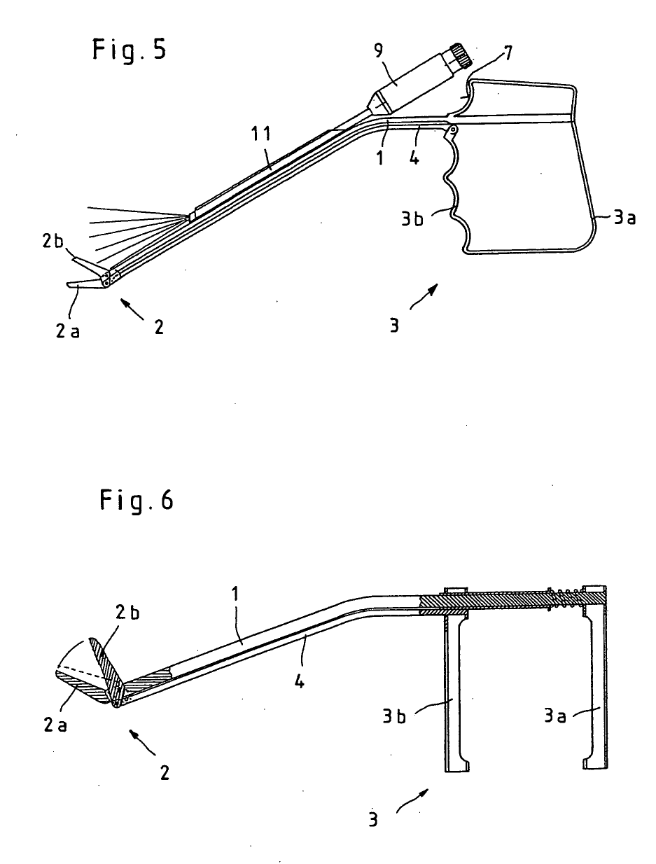

In addition to the varied manner of mounting the movable gripping member 3b of the handle 3, i.e. as a rotatable gripping member 3b as in FIGS. 1a to 4, or as a gripping member 3b that can slide axially as in FIGS. 5 and 6, the embodiments of medical cutting and/or holding instruments depicted in FIGS. 1a to 6 are distinguished essentially by the shape and length of configuration of the shafts 1 and the push-pull rods 4.

In the first embodiment, depicted in FIG. 1a, the push-pull rod 4 and the shaft 1 each run parallel to one another with a bend downward, with the push-pull rod 4 in this embodiment situated below the shaft 1.

The gripping member 3b, which can rotate around the pivot axis 5 positioned on the rigid gripping member 3a, can be fixed by a preferably spring-weighted stopping device 6 in the position that can rotate with respect to the rigid gripping member 3a. As can further be seen from FIG. 1a, on the rigid gripping member 3a of the handle 3 a support 7 for the index finger of the user of the medical instrument is configured in order to be able to compensate for the thrust usually caused in open surgery in activating the movable gripping member 3b. This index finger support 7, independent of the movable gripping member 3b, in addition to supporting the hand on the rigid gripping member 3a of the handle 3, allows a gentle, stable activation of the medical instrument.

The embodiments illustrated in FIGS. 1a and 1b are distinguished by the orientation of the jaw members 2a and 2b of the tool 2 to the longitudinal axis of the shaft as well as to the push-pull rod 4. Whereas in the embodiment in FIG. 1a the jaw members 2a and 2b of the tool 2 are clearly configured as bent with respect to the longitudinal axis, in the embodiment in FIG. 1b they run essentially in the axial direction.

In the second embodiment shown in FIG. 2 the push-pull rod 4 and shaft 1 run only partially parallel to one another, so that the push-pull rod 4 in this embodiment is situated above the shaft 1.

The gripping member 3b that can rotate around the pivot axis 5 on the shaft 1, in turn, can be fixed in the position rotated with respect to the rigid gripping member 3a by means of a spring-loaded stopping device 6. As further to be seen from FIG. 2, in this embodiment the support 7 for the user's index finger and the stopping device 6 are configured as a single piece so that the stopping device 6 is deactivated when the index finger is applied, but upon releasing the index finger support 7 the stopping device 6 immediately engages as a block on the rigid gripping member 3a because of the spring loading. To make room for the index finger support 7, the shaft 1 at this site is configured as strongly bent downward from the push-pull rod 4.

In addition, the handle 3 includes a spring element 8 by means of which the rotatable gripping member 3b is pre-tensioned in the position that opens the tool 2.

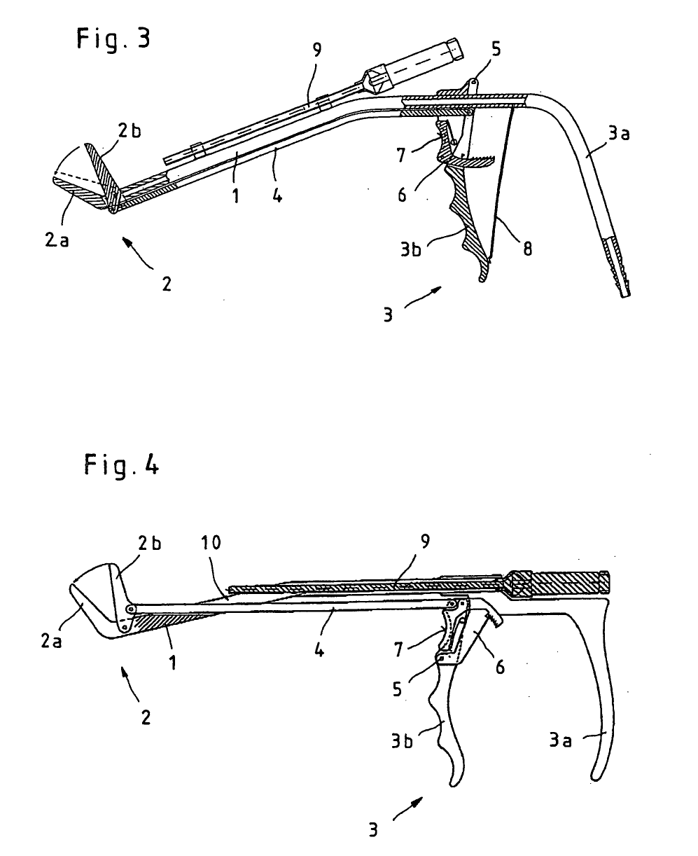

In the third embodiment, illustrated in FIG. 3, the push-pull rod 4 and the shaft 1, as already described in relation to FIG. 1a, run parallel to one another, each bent downward, so that the push-pull rod 4 is positioned below the shaft 1.

The gripping member 3b, which can rotate around the pivot axis 5 on the shaft 1 and is pre-tensioned by the spring element 8 in the open direction of the tool 2, can also be fixed by a spring-loaded stopping device 6 in this embodiment in the position that is rotatable with respect to the rigid gripping member 3a, so that the support 7 for the user's index finger and the stopping device 6 are configured as a single piece in the manner described previously in relation to the second embodiment, shown in FIG. 2.

The instruments shown in FIGS. 2 and 3 are distinguished in addition in that the rigid gripping member 3a and the shaft 1 are configured as a one-piece component so that assembly and disassembly of the instrument can be considerably simplified.

In order to improve viewing conditions for the user in narrower operating passages, on the shaft 1 an illumination element 9 can be secured which preferably has its own power supply by means of a battery or accumulator battery. In addition to the embodiment shown in FIG. 3, in which the illumination element 9 can be secured on the shaft 1, it is also possible of course to position the illumination element 9 on the push-pull rod 4, especially if this rod, as seen in FIG. 2, is positioned above the shaft 1.

In the fourth embodiment, shown in FIG. 4, the course of the straight push-pull rod 4 and of the shaft 1, simply turned downward, cross one another. This embodiment includes a breech 10, to permit the push-pull rod 4 to pass through the shaft so that the push-pull rod 4 is positioned below the shaft 1 on the rotatable gripping member 3b, but is positioned above the shaft 1 on the rotatable jaw member 2b.

The gripping member 3b, which can rotate around the pivot axis 5 mounted on the rigid gripping member 3a, can also be fixed, in this embodiment, by a spring-weighted stopping device 6 in the position that can rotate with respect to the rigid gripping member 3a, so that the support 7 for the user's index finger and the stopping device 6 are configured as a single unit as described previously in relation to the second embodiment, illustrated in FIG. 2.

This illustrated embodiment also shows the use of an illumination element 9 that can be secured on the instrument. Even if not all the illustrated medical instruments are equipped with an illumination element 9, with all instruments there is the possibility of providing them with an illumination element 9 of this type if required.

The fifth and sixth embodiments, illustrated in FIGS. 5 and 6, finally, show a medical cutting and/or holding instrument with an axially slidable gripping member 3b.

In these two embodiments, the push-pull rod 4 and the shaft 1 run parallel to one another, each bent downward, so that the push-pull rod 4 is situated below the shaft 1 in each case. Of course, with this type of configuration of the movable gripping member 3b, it is also possible to design the structure in such a way that the push-pull rod 4 is positioned in each case above the shaft 1.

The embodiment shown in FIG. 5 is further distinguished in that the axially slidable gripping member 3b is configured as a one-piece unit with the rigid gripping member 3a and the ability of the gripping member 3b to be displaced depends on the spring elasticity of the handle material.

For housing the illumination element 9, the medical instrument shown in FIG. 5 has on the upper side of the shaft 1 a channel 11, into which the illumination element 9 can be inserted.

The medical cutting and/or holding instruments configured according to the illustrations are distinguished in that on the one hand the push-pull rods 4, which are configured independently of the shafts 1, can be cleaned and assembled easily and quickly and on the other hand the shafts 1 and push-pull rods 4 can be adapted individually to the immediate operating purpose. The medical instruments configured in this way are appropriate for use in open surgery because of the simple and robust structure.

1. A medical cutting and/or holding instrument having a shaft, a handle consisting of two gripping members on the proximal end of the shaft, and a push-pull rod for activating a tool that is positioned on the distal end of the shaft and consists of two jaw members, such that the push-pull rod can be coupled with at least one rotatable jaw member of the tool on the distal end and with one movable gripping member of the handle on the proximal end for opening and closing the tool, characterized in that the push-pull rod is configured as a component that is independent of the shaft and is positioned outside the shaft, exclusively on the rotatable jaw member of the tool and on the movable gripping member of the handle.

2. A medical cutting and/or holding instrument according to claim 1, characterized in that the push-pull rod and the shaft are configured differently in terms of shape and/or length.

3. A medical cutting and/or holding instrument according to claim 1, characterized in that the push-pull rod and/or the shaft has at least one bend with respect to the longitudinal axis.

4. A medical cutting and/or holding instrument according to claim 1, characterized in that there is a breech for passage of the respective other component configured in the shaft or in the push-pull rod.

5. A medical cutting and/or holding instrument according to claim 1, characterized in that at least one channel running in the axial direction is configured in or on the shaft and/or the push-pull rod.

6. A medical cutting and/or holding instrument according to claim 1, characterized in that an illumination element can be secured on the shaft or on the push-pull rod.

7. A medical cutting and/or holding instrument according to claim 1, characterized in that the push-pull rod can be dissolubly secured on the proximal end on the movable gripping member by means of a coupling mechanism.

8. A medical cutting and/or holding instrument according to claim 1, characterized in that the coupling mechanism is configured as a screw-in connection.

9. A medical cutting and/or holding instrument according to claim 1, characterized in that the movable gripping member of the handle is configured as a gripping member that can rotate around a pivot axis.

10. A medical cutting and/or holding instrument according to claim 1, characterized in that the movable gripping member of the handle is configured as a gripping member that can slide axially in the direction of the push-pull rod.

11. A medical cutting and/or holding instrument according to claim 1, characterized in that the non-movable gripping member is configured as a single piece with the shaft 1.

12. A medical cutting and/or holding instrument according to claim 1, characterized in that a support for the user's index finger, independent of the movable gripping member, is configured on the handle.
